(19) United States
(12) Patent Application Publication
(10) Pub. No.: US 2023/0341374 A1
(43) Pub. Date: Oct. 26, 2023

(54) Rapid bioaerosol detection method in exhaled breath

(71) Applicants: Anindya Chanda, North Potomac, MD (US); Gregory M. Gandee, Mason Neck, VA (US)

(72) Inventors: Anindya Chanda, North Potomac, MD (US); Gregory M. Gandee, Mason Neck, VA (US)

(21) Appl. No.: 18/303,667

(22) Filed: Apr. 20, 2023

Related U.S. Application Data
(60) Provisional application No. 63/334,879, filed on Apr. 26, 2022.

(51) Int. Cl. G01N 33/497; G01N 15/14

(52) U.S. Cl. CPC G01N 33/497; G01N 15/14; G01N 2033/4975

(57) ABSTRACT

A method to determine whether a person is non-infectious for an infectious disease that transmits from person to person via exhaled bioaerosols comprises measuring viral and bacterial load in exhaled breath of an individual using a non-invasive instrument to detect levels of exhaled viral and bacterial loads.

### CROSS-REFERENCE TO RELATED APPLICATIONS

This application claims the benefit of U.S. Provisional Patent Application No. 63/334,879 filed on Apr. 26, 2022, the entire contents of which is incorporated herein by reference.

### TECHNICAL FIELD

The present disclosure describes a non-invasive, rapid detection method for determining whether a person is non-infectious for an infectious disease that transmits from person to person via exhaled bioaerosols.

### BACKGROUND

Mitigating the spread of an infectious disease that transmits from person to person via exhaled bioaerosols is of vital importance for many reasons, including for example, public health, economic, educational, and political reasons. The recent SARS-Cov-2 pandemic showed that restoring normal business operations and normal primary education operations can be challenging because there is currently no available method to quickly and accurately determine and identify individuals who are non-infectious and therefore pose little to no risk of spreading an infectious disease via exhaled bioaerosols. When returning to school, work, and other locations of gathering during the SARS-Cov-2 pandemic, body temperature measurements were used as a rapid screening procedure to determine who could be considered negative for the virus and therefore able to be admitted into the relevant location (e.g., school, work, daycare, etc.). Unfortunately, body temperature screening has proven ineffective for asymptomatic individuals. Accordingly, an accurate and rapid screening method for determining who is non-infectious for an infectious disease that transmits from person to person via exhaled bioaerosols is needed.

### BRIEF DESCRIPTION OF THE FIGURES

The foregoing aspects and other features of the disclosure are explained in the following description, taken in connection with the accompanying drawings, wherein:

FIG. 1 is a schematic flow chart illustrating an embodiment of how the bioaerosol sensing device described herein can be used.

FIG. 2A is a bar chart showing exemplary readings from the bioaerosol sensing device.

FIG. 2B is a bar chart showing exemplary readings from the bioaerosol sensing device.

FIG. 3A is an additional bar chart showing additional exemplary readings from the bioaerosol sensing device.

FIG. 3B is an additional bar chart showing additional exemplary readings from the bioaerosol sensing device.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “a composite” means at least one composite and can include more than one composite.

Throughout the specification, the terms “about” and/or “approximately” may be used in conjunction with numerical values and/or ranges. The term “about” is understood to mean those values near to a recited value. For example, “about 40 [units]” may mean within +/−25% of 40 (e.g., from 30 to 50), within +/−20%, +/−15%, +/−10%, +/−9%, +/−8%, +/−7%, +/−6%, +/−5%, +/−4%, +/−3%, +/−2%, +/−1%, less than +/−1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

Throughout the specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

As used herein, the verb “comprise” as is used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

Described herein is a rapid method to determine whether a person is non-infectious for an infectious disease that transmits from person to person via exhaled bioaerosols. The method uses a non-invasive, non-specific, pre-screening test to detect levels of exhaled viral and bacterial loads. The method is not specific to a particular infectious disease. Rather, the method can detect total viral and bacterial loads in exhaled air thereby quickly determining whether a person has a relatively high viral or bacterial load in his or her exhaled air. In embodiments of the method, the test can be used to pre-screen individuals to determine whether their viral and bacterial load is below a predetermined “safe level.” For example, the method could be used as a pre-screening test prior to entry into a school, public transportation, a restaurant, an event, etc. The described method will allow direct relative quantification of exhaled viral clumps in a time span of about 8 seconds.

The method described herein is more reliable and accurate than measuring body temperature, which is the current rapid pre-screening method for SARS-Cov-2. Currently, there are no available methods that can determine real-time if an asymptomatic individual entering a public area has the potential to infect others via normal activity, e.g., breathing, talking, shouting, singing, etc.

By quickly determining viral levels in exhaled breath, the described method can determine with >99% certainty whether the person being screened is non-infectious. With this method, businesses, schools, and other public venues can operate with more piece of mind with the knowledge that individuals having viral and/or bacterial loads indicative of infectious disease have been identified.

Threshold levels indicative of viral and bacterial load for individuals that are not infectious can be determined. For example, a viral load of 100,000 count/cubic meter of the number of total viral clumps (>0.5 μm) could be a threshold level. Exemplary alternative threshold viral loads could be 90,000 count/cubic meter; 80,000 count/cubic meter; 70,000 count/cubic meter; 60,000 count/cubic meter; etc. If the exhaled breath of a person shows a viral load of less than the threshold level, that individual would be deemed non-infectious and would pass through the so-called pre-screening. With the method described herein, an individual's viral load can be determined rapidly and easily, for example, within about 8-10 seconds.

The bioaerosol screening method employs an instrument, for example, a cytometer, implemented with a breathing tube into which a subject can blow exhaled breath. The instrument is equipped with a microbial sensor for relative quantification of viral and bacterial clumps (>0.5 μm) in bioaerosols exhaled from the mouth. Upon exhalation of a subject into the breathing tube, the cytometer can determine the amount of viral and/or bacterial clumps in the exhaled breath in real-time. In embodiments, the breathing tube can be disposable, such as, for example, a disposable straw. The instrument can directly analyze a sample of air breathed out and quickly determine if the individual is shedding higher than non-infectious levels of virus and bacteria. The method can screen-out an individual who is asymptomatic with normal body temperature, but who is shedding viral particles at quantities high enough to infect others.

In embodiments, the instrument can include a fluorescence-based direct-reading cytometer. An exemplary instrument is an InstaScope, which is manufactured by DetectionTek, Boulder, CO, USA. An InstaScope can be equipped with an optical reference library of biological signatures to characterize airborne bioaerosols. The device can be calibrated before sampling. The InstaScope can include a mobile version of a Wideband Integrated Bioaerosol Sensor (WIBS), also manufactured by DetectionTek, Boulder, CO, USA.

To perform the method described herein, the instrument must be modified to include a breathing tube into which a subject can exhale breathing air to enable detection and determination of viral and/or bacterial load. Devices enabling real-time, accurate viral and bacterial load detection and quantification in breath exhaled from a single individual are currently unavailable.

In an exemplary embodiment, an InstaScope can sample particles that enter the instrument through an inlet wand and enter a chamber equipped with a continuous diode laser to produce scattered light, which Xenon flashlamps then detect. Upon detection, if particles emit fluorescence due to excitation, two photomultiplier tubes designed to detect such emissions then log total counts, particle size, and fluorescence properties. The particles can be characterized as one of seven types first introduced by Perring et al. which considered three fluorescence bandwidths individually and in all possible combinations.

The WIBS instrument can use two excitation wavelengths, 280 and 370 nm. Fluorescence emitted by particles can be recorded by three channels (Channel A, Channel B, and Channel C). The notations TYPE A280=(310-400), TYPE B280=(420-650), and TYPE C370=(420-650) can be utilized. Each subscripted integer denotes excitation wavelengths, while the parentheses indicate the emission bandwidths observed. Any particle could have signaled above the fluorescence threshold within these channels, leading to seven (A, B, C, AB, AC, BC, and ABC) likely combinations of fluorescence signal. Bacterial, fungal, and pollen particle classification based on fluorescence and channel type are also described in the literature.

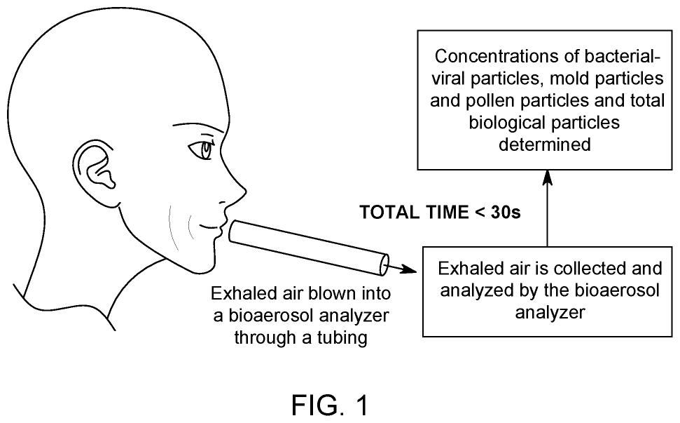

The method described herein is a non-invasive method of quantifying the amounts of different biological particles in bioaerosols released in exhaled air. FIG. 1 is a schematic flow chart that shows and explains how the method can be performed. A subject exhales air in a breathing tube, which can be a disposable breathing tube, such as, for example, a straw. The exhaled air enters the instrument through the breathing tube. An exemplary instrument is the InstaScope, which is manufactured by DetectionTek, Boulder, CO, USA. The exhaled air is collected and analyzed by the InstaScope bioaerosol analyzer. Concentrations of bacterial and viral particles, mold particles and pollen particles, and total biological particles is determined.

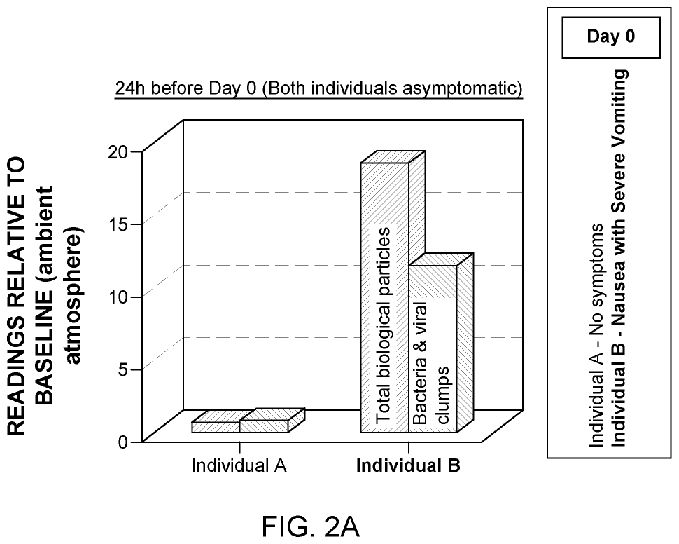

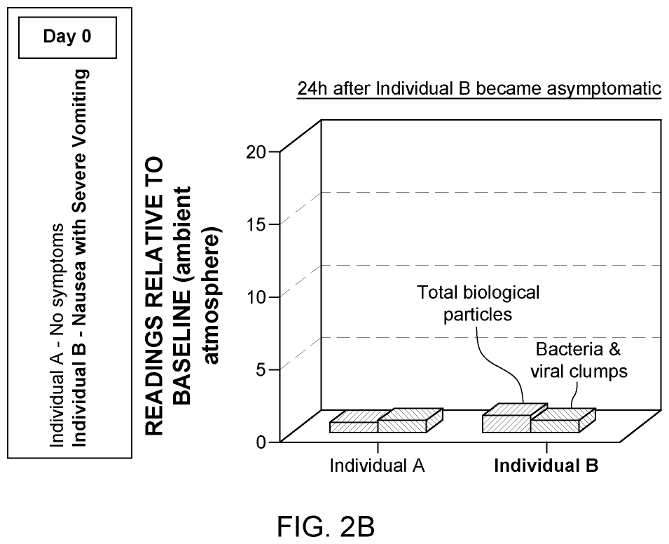

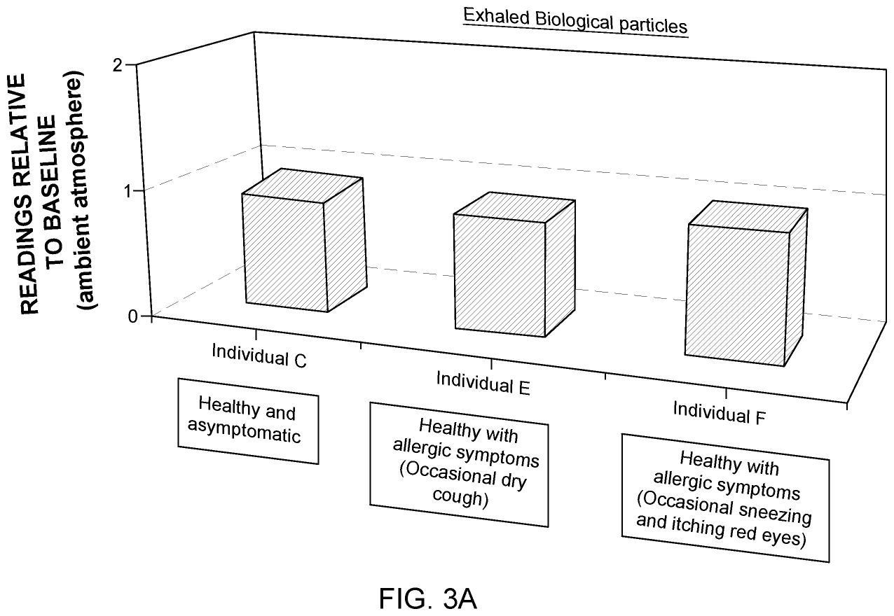

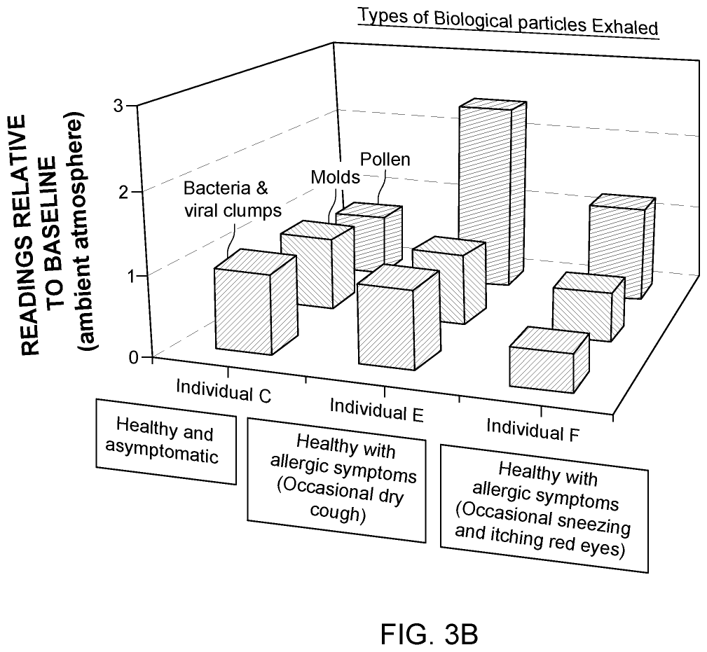

Testing has been performed to determine efficacy of the method described herein. FIG. 2A, FIG. 2B, FIG. 3A and FIG. 3B are bar charts showing the results of testing. As can be seen in FIG. 2A and FIG. 2B, the bioaerosol exhalation method can determine whether an asymptomatic individual has a bacterial and viral load in exhaled breath that is indicative of the individual shedding higher than normal bacterial and viral load 24 hours before the individual begins exhibiting symptoms of viral or bacterial infection. Thus, the described method can identify individuals shedding relatively high levels of viral and bacterial load prior to the individual becoming symptomatic. The method is also able to show when an individual is shedding a more normal, non-infectious viral and bacterial load through exhaled breath. In FIG. 2A and FIG. 2B, if the method were used as a pre-screening method to determine which individuals were infectious and which individuals were not infectious, for FIG. 2A, Individual A would be non-infectious and Individual B would be infectious. For FIG. 2B, both Individual A and Individual B would be non-infectious.

FIG. 3A shows total exhaled biological particles and FIG. 3B shows exhaled biological particles broken down into types of biological particles exhaled by three individuals. In FIG. 3A, all three individuals have loads of biological particles indicative of healthy subjects. FIG. 3B provides a more detailed analysis of the exhaled bioaerosol particles. Exhaled particles for Individual C indicate that the subject was healthy and asymptomatic. Exhaled particles for Individual E indicate that the subject was healthy with allergic symptoms. Exhaled particles for Individual F indicate that the subject was healthy with allergic symptoms.

### Example

Testing will be performed to determine threshold levels that can be used for pre-screening. Individuals will be asked to take one deep breath and exhale that breath during a span of 8s into a disposable straw. The exhaled breath will be analyzed by the InstaScope. The exhalation will be performed 3 times. The relative ratio of viral emission (RVE) calculated as viral load of the individual/average viral load of the ambient will be determined for each individual test run. The instrument will report as (count/cubic meter) the number of total viral clumps (>0.5 μm) in bioaerosols released in exhaled breath and provides a report in count/cubic meter air unit.

The individuals will also take a Covid PCR test to determine which individuals are Covid negative and which individuals are Covid positive. The Covid test results will be cross referenced with the results of the exhaled breathing analysis to determine which RVEs correspond to Covid negative results and which RVEs correspond to Covid positive results.

### REFERENCES

Nieto-Caballero, M.; Gomez, O. M.; Shaughnessy, R.; Hernandez, M. Aerosol fluorescence, airborne hexosaminidase, and quantitative genomics distinguish reductions in airborne fungal loads following major school renovations. Indoor Air 2022
Hernandez, M.; Perring, A. E.; McCabe, K.; Kok, G.; Granger, G.; Baumgardner, D. Chamber catalogues of optical and fluorescent signatures distinguish bioaerosol classes. Atmos. Meas. Tech. 2016, 9, 3283-3292.
Perring, A. E.; Schwarz, J. P.; Baumgardner, D.; Hernandez, M. T.; Spracklen, D. V.; Heald, C. L.; Gao, R. S.; Kok, G.; McMeeking, G.; McQuaid, J. B.; et al. Airborne observations of regional variation in fluorescent aerosol across the United States. J. Geophys. Res. Atmos. 2015, 120, 1153-1170.
Handorean, A.; Robertson, C. E.; Harris, J.; Frank, D. N.; Hull, N. M.; Kotter, C.; Stevens, M. J.; Baumgardner, D.; Pace, N. R.; Hernandez, M. Microbial aerosol liberation from soiled textiles isolated during routine residuals handling in a modern health care setting. Microbiome 2015, 3, 72.
Kaye, P. H.; Stanley, W.; Hirst, E.; Foot, E. V.; Baxter, K. L.; Barrington, S. J. Single particle multichannel bio-aerosol fluorescence sensor. Opt. Express 2005, 13, 3583-3593.
Robinson, E. S.; Gao, R.-S.; Schwarz, J. P.; Fahey, D. W.; Perring, A. E. Fluorescence calibration method for single-particle aerosol fluorescence instruments. Atmos. Meas. Tech. 2017, 10, 1755-1768.

That which is claimed:

1. A method to determine whether a person is non-infectious for an infectious disease that transmits from person to person via exhaled bioaerosols comprising measuring viral and bacterial load in exhaled breath of an individual using a non-invasive instrument to detect levels of exhaled viral and bacterial loads.

2. The method of claim 1, wherein the method is not specific to a particular infectious disease.

3. The method of claim 1, wherein Rather, the method detects viral and bacterial loads in exhaled air thereby quickly determining whether a person has a relatively high viral or bacterial load in his or her exhaled air.

4. The method of claim 1, wherein the method is used to pre-screen individuals to determine whether their viral and bacterial load is below a predetermined threshold level.

5. The method of claim 1, wherein the method is used as a pre-screening test prior to entry into a school, public transportation, a restaurant, an event, etc.

6. The method of claim 1, wherein relative quantification of exhaled viral clumps is performed in a time span of about 8 seconds.
